(19) United States
(12) Patent Application Publication
(10) Pub. No.: US 2023/0110651 A1
(43) Pub. Date: Apr. 13, 2023

(54) ASSAYS TO QUANTITATE DRUG AND TARGET CONCENTRATIONS

(71) Applicant: Regeneron Pharmaceuticals, Inc., Tarrytown, NY (US)

(72) Inventors: Jihua Chen, Ossining, NY (US); Kimberly Kendra, Carmel, NY (US); Stacey Shank, Southbury, CT (US); Lisa DeStefano, Yorktown Heights, NY (US); Matthew Andisik, Yorktown Heights, NY (US); Michael Partridge, Eastchester, NY (US); Albert Torri, LaGrangeville, NY (US); Giane Oliveira Sumner, Mahwah, NJ (US)

(21) Appl. No.: 17/954,999

(22) Filed: Sep. 28, 2022

Related U.S. Application Data
(60) Provisional application No. 63/249,417, filed on Sep. 28, 2021.

(51) Int. Cl. G01N 33/94; G01N 33/58

(52) U.S. Cl. CPC G01N 33/94; G01N 33/581; G01N 2470/04

(57) ABSTRACT

The present invention generally pertains to methods of determining concentrations of drugs and their targets. In particular, the present invention, in part, pertains to use of a mild acidic assay condition to determine total drug and total target concentrations to mitigate either target interference or drug interference. The present invention also discloses a free target assay using a capture agent that has a lower affinity with a much slower association and dissociation rate compared to the drug.

### CROSS-REFERENCE TO RELATED APPLICATIONS

This application claims priority to and the benefit of U.S. Provisional Pat. Application No. 63/249,417, filed Sep. 28, 2021, which is herein incorporated by reference.

### FIELD

The present invention generally pertains to providing total drug assays, free target assays and total target assays, which can accurately quantify drug and target concentrations in clinical study samples.

### BACKGROUND

The measurement of low amounts of biotherapeutic drug in complex biological samples, such as serum, is of growing clinical importance for patient management, as well as basic science. For example, monoclonal antibodies (mAbs) are a fast-growing class of biotherapeutics for the treatment of a variety of conditions such as diabetes, cancers, inflammation and infectious diseases, etc. Ligand-binding assays (LBA) are commonly used to quantitate mAbs and the corresponding targets. Multiple forms of mAbs and targets exist in biological samples, including free mAbs (i.e., unbound to target), free targets (i.e., unbound to mAbs or soluble physiological/endogenous binding partners), and monovalent and/or bivalent complexes of mAbs and targets. In the nonclinical setting, total mAb concentrations in circulation are usually used to evaluate systemic exposure and evaluate potential drug toxicity to help determine a safe starting dose and/or efficacious dose in first-in-human (FIH) studies.

During clinical evaluations, mAb concentration data are used to determine key pharmacokinetic (PK) parameters, to characterize drug disposition, correlate exposure with safety and efficacy, and to provide dosing regimen selections for later stage studies. Target concentrations can also be used in the nonclinical development stage to determine the efficacious mAb concentration and to allow the model-based determination of dose. Target data in the clinical setting can be used to characterize human PK profiles, define PK/pharmacodynamic (PD) relationships regarding safety and efficacy, and establish PK/PD models in the disease population. In particular, total target data provides information on the effect of mAbs on target accumulation and whether there is continuous target engagement in circulation to achieve sustained complete target suppression. Free target data are informative for determining the efficacious dose and guiding dose level/schedule selection. Free targets can also be used for PK/PD modeling and to help understanding other PD/end point results.

Although the measurement of the drug and the target can provide important information, the accuracy of drug and target measurement depends on the appropriate design of the bioanalytical methods and the quality of the critical reagents employed. Capture and detection reagents are critical components in determining assay specificity for free and total target assays. Furthermore, the presence of the biotherapeutic drug may interfere with the accurate quantification of the total targets if the anti-target antibodies used in the target assays have overlapping regions of recognition with the biotherapeutic drug. Therefore, certain strategies, including the use of acid dissociation and anti-drug antibodies, need to be used to mitigate drug interference. In addition, anti-target antibodies that do not compete with drugs for target binding can be used as the capture and detection reagents. However, the drug in the target: drug complexes may still impact the accurate detection of the total target due to steric hindrance. Assay pH can also be adjusted to selectively alter the binding of target to the drug and/or to the capture and detection reagents to mitigate drug interference.

Developing a highly sensitive LBA method for free target measurement in the presence of the drug is also challenging because in many cases, free target concentrations are very low, with rapid turnover and may vary with assay conditions. As the capture antibody usually competes for the same binding epitope on the target as the drug, shifts in sample equilibrium between the drug-bound target and the capture antibody during the assay process can lead to overestimation of free target concentrations. Therefore, it is important to fully understand the dynamics of the association of the biotherapeutic drug and target. The assay conditions should be optimized to minimize the dissociation of existing drug-bound target and accurately measure the free target. The affinity of the capture antibody to the target usually needs to be much weaker compared with that of the drug to minimize any impact in the sample equilibrium, and any artificial increase in concentrations of free target. For similar reasons, the concentration of the capture antibody should be optimized. In addition, other factors such as sample collection, dilution, freeze/thaw cycles, and storage may also shift the dynamics of the association between the biotherapeutic drug and target; for example, use of buffers with high salt or detergent concentrations and long sample incubation times may affect the dissociation between drug and target.

A free target assay can also be developed by removing the bound target with immunoprecipitation, solid phase extraction, or affinity separation. However, the additional processes may introduce other variability due to adsorption of target to the column or filter/beads surface. Dissociation of the target:drug complexes may still occur during the whole process. In addition, the procedures are labor-intensive with low throughput and require rigorous standardization of each individual assay procedure step. Other assay platforms such as Gyrolab technology can also be used to enable quantitation of free target usually with minimal sample dilution and short sample incubation time. See Dysinger and Ma. AAPS J., 20(6), 106 (2018).

Finally, the development of a total drug assay can also be challenging, especially when the target is present at a high concentration, which may interfere with the ability of the anti-idiotype capture or detection mAb to bind the drug. See Lee, J.W., et al., AAPS J, 2011. 13(1): p. 99-110; Watanabe et al. AAPS J. 23(1), 21 (2021).

LC-MS has emerged as a quantitative tool to measure the concentrations of biotherapeutic drugs and their targets. See van de Merbel NC. Bioanalysis 11(7), 629-644 (2019). It provides a wide dynamic range with good accuracy and precision. It may also be used to quantitate multiple analytes simultaneously and is less dependent on critical immunoreagents. This approach can also overcome assay interference from, for example, drug, target and other endogenous binding proteins, and it can be easily used to measure total drug or target. However, LC-MS is typically a low-throughput method with limited sensitivity.

Since accurate quantitation of therapeutic proteins and their targets is critical for the assessment of exposure-response relationships in support of efficacy and safety evaluations and dose selection. Therefore, it is important to develop reliable and accurate bioanalytical methods to support the drug development program.

### SUMMARY

Exemplary embodiments disclosed herein satisfy the aforementioned demands by providing methods for determining drug and target concentrations.

This disclosure provides a method for determining concentration of free target in a sample. In one exemplary embodiment, the method comprises adding said sample having a bound target and said free target to a solid support coated with a capture agent, wherein said capture agent has less affinity and a much slower association rate for the target compared to the drug; adding a detection agent with a detectable label; and measuring a signal from the detectable label of the detection agent to determine said concentration of free target in said sample, whereby the signal is proportional to the concentration of said free target in said sample.

In one aspect of this embodiment, the method further comprises determining an amount of free target from said signal by comparing it to a standard calibration curve, wherein the standard calibration curve is produced by carrying out the method by using at least three standard solutions having three different concentrations of said free target instead of said sample.

In one aspect of this embodiment, the sample is incubated with the capture agent for about 15 minutes. In another aspect of this embodiment, the sample is incubated with the capture agent for about 30 minutes. In yet another aspect of this embodiment, the sample is incubated with the capture agent for about 45 minutes. In yet another aspect of this embodiment, the sample is incubated with the capture agent for about 60 minutes.

In one aspect of this embodiment, the sample is incubated with the detection agent for about 15 minutes. In another aspect of this embodiment, the sample is incubated with the detection agent for about 30 minutes. In yet another aspect of this embodiment, the sample is incubated with the detection agent for about 45 minutes. In yet another aspect of this embodiment, the sample is incubated with the detection agent for about 60 minutes.

In one aspect of this embodiment, affinity of said capture agent is less than the drug by about 2-fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, or about 50-fold. In another aspect of this embodiment, affinity of said capture agent is less than the drug by more than about 50-fold.

In one aspect of this embodiment, half-life of said capture agent is more than the drug by about 2 times, about 3 times, about 4 times, about 5 times, about 6 times, about 7 times, about 8 times, about 9 times, or about 10 times. In another aspect of this embodiment, half-life of said capture agent is more than the drug by more than about 10 times.

In one aspect of this embodiment, the solid support is a streptavidin coated.

In one aspect of this embodiment, the capture agent is biotinylated.

In one aspect of this embodiment, the detectable label is ruthenium. In another aspect of this embodiment, the detectable label is an electrochemiluminescent substrate.

In one aspect of this embodiment, said signal is obtained by applying voltage.

In one aspect of this embodiment, said detection agent is different from said capturing agent. In another aspect of this embodiment, said detection agent is the same as said capturing agent.

This disclosure provides a method for determining concentration of total target in a sample. In one exemplary embodiment, the method comprises contacting said sample to an acid solution; adding said sample to a solid support coated with a capture agent; adding a detection agent with a detectable label; measuring a signal from the detection agent to determine the concentration of said total target in said sample, wherein said total target includes target complexed with a drug and free target, and whereby the signal is proportional to the concentration of said total target in said sample.

In one aspect of this embodiment, the method further comprises determining an amount of total target from said signal by comparing it to a standard calibration curve, wherein the standard calibration curve is produced by carrying out the method by using at least three standard solutions having three different concentrations of free target instead of said sample.

In one aspect of this embodiment, said signal is obtained by applying voltage.

In one aspect of this embodiment, said acid solution has a pH of about 5.0 to about 7.0. In a preferred embodiment, said acid solution has a pH of about 6.0.

In one aspect of this embodiment, said acid solution comprises about 50-500 mM acetic acid. In a preferred embodiment, said acid solution comprises about 300 mM acetic acid. In another preferred embodiment, said acid solution comprises about 30 mM acetic acid.

In one aspect of this embodiment, said capture agent has a lower dissociation rate and greater t_{½} towards said target than said drug.

In one aspect of this embodiment, the dissociation rate of said capture agent is lower than the drug by about 2-fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, or about 50-fold. In another aspect of this embodiment, the dissociation rate of said capture agent is lower than said drug by more than about 50-fold.

In one aspect of this embodiment, t_{½} of said capture agent is more than the drug by about 2 times, about 3 times, about 4 times, about 5 times, about 6 times, about 7 times, about 8 times, about 9 times, or about 10 times. In another aspect of this embodiment, t_{½}of said capture agent is more than the drug by more than about 10 times.

In one aspect of this embodiment, said detection agent has a lower dissociation rate and greater t_{½} towards said target than said drug.

In one aspect of this embodiment, the dissociation rate of said detection agent is lower than the drug by about 2-fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, or about 50-fold. In another aspect of this embodiment, the dissociation rate of said detection agent is lower than the drug by more than about 50-fold.

In one aspect of this embodiment, t_{½}of said detection agent is more than the drug by about 2 times, about 3 times, about 4 times, about 5 times, about 6 times, about 7 times, about 8 times, about 9 times , or about 10 times. In another aspect of this embodiment, t_{½} of said detection agent is more than the drug by more than about 10 times.

In one aspect of this embodiment, the sample is incubated with the capture agent for about 15 minutes. In another aspect of this embodiment, the sample is incubated with the capture agent for about 30 minutes. In yet another aspect of this embodiment, the sample is incubated with the capture agent for about 45 minutes. In yet another aspect of this embodiment, the sample is incubated with the capture agent for about 60 minutes.

In one aspect of this embodiment, the sample is incubated with the detection agent for about 15 minutes. In another aspect of this embodiment, the sample is incubated with the detection agent for about 30 minutes. In yet another aspect of this embodiment, the sample is incubated with the detection agent for about 45 minutes. In yet another aspect of this embodiment, the sample is incubated with the detection agent for about 60 minutes.

In one aspect of this embodiment, said detection agent is different from said capturing agent. In another aspect of this embodiment, said detection agent is the same as said capturing agent.

In one aspect of this embodiment, the solid support is a streptavidin coated.

In one aspect of this embodiment, the capture agent is biotinylated.

In one aspect of this embodiment, the detectable label is ruthenium. In another aspect of this embodiment, the detectable label is an electrochemiluminescent substrate.

This disclosure provides a method for determining concentration of total drug in a sample. In one exemplary embodiment, the method comprises adding said sample to a solid support coated with a capture agent; adding a detection agent with a detectable label, wherein said capture agent is different than the capture agent; and measuring a signal from the detection agent to determine the concentration of said total target in said sample, wherein said total drug includes drug complexed to a target and free drug, and whereby the signal is proportional to the concentration of said free target in said sample.

In one aspect of this embodiment, the method further comprises adding a substrate specific to binding with the detection agent, wherein said substrate bound to the detection agent provides said signal.

In one aspect of this embodiment, the capture agent is a monoclonal antibody that binds to said drug.

In one aspect of this embodiment, said detection agent is different from said capturing agent.

In one aspect of this embodiment, wherein the detection agent is biotinylated.

In one aspect of this embodiment, said acid solution has a pH of about 5.0 to about 7.0. In a preferred embodiment, said acid solution has a pH of about 6.0.

In one aspect of this embodiment, said acid solution comprises about 50-500 mM acetic acid. In a preferred embodiment, said acid solution comprises about 300 mM acetic acid. In another preferred embodiment, said acid solution comprises about 30 mM acetic acid.

### BRIEF DESCRIPTION OF THE DRAWINGS

FIG. 1A shows a standard curve signal with neutral buffer (assay dilution buffer, no acid), acid treatment (300 mM, pH ∼ 3.2) with neutralization and mild acid (30 mM), according to an exemplary embodiment.

FIG. 1B shows an assay signal of LLOQ spiked samples with acid treatment (300 mM) and with mild acid (30 mM), according to an exemplary embodiment.

FIG. 2A shows total target levels in three human plasma samples in the absence and presence of 1 mg/mL of drug under neutral assay pH, according to an exemplary embodiment.

FIG. 2B shows total target assay signal with neutral assay pH, and with acid treatment (300 mM acetic acid, pH ~3.2) and neutralization, and with 30 mM acetic acid in 5% BSA (pH ~6.0), according to an exemplary embodiment.

FIG. 2C shows total target levels in four human plasma samples in the absence and presence of 1 mg/mL of drug under mild acidic assay pH (pH ~6.0), according to an exemplary embodiment.

FIG. 2D shows total target concentrations in four human plasma samples with or without 1 mg/mL of drug, in the presence of 200 µg/mL or 1 mg/mL of anti-drug antibody blocker under mild acidic assay pH (pH ~6.0), according to an exemplary embodiment.

FIG. 2E shows total target levels in three human plasma samples with or without 1 mg/mL of drug with the new capture and detection antibody pair and with mild assay pH (pH ~ 6.0), according to an exemplary embodiment.

FIG. 3A shows LLOQ signal from the free target assay and the assay signal from the target:drug complexes in a 1:5, 1:2 and 1:1 molar ratio, with a 1:2 sample dilution in 5% BSA, and with Mab-2 or Mab-3 as the capture antibody, according to an exemplary embodiment.

FIG. 3B shows LLOQ signal from the free target assay and the assay signal from the target:drug complexes in a 1:5, 1:2 and 1:1 molar ratio, with a 1:50 sample dilution in 5% BSA, and with Mab-2 or Mab-3 as the capture antibody, according to an exemplary embodiment.

FIG. 3C shows Predicted (green bars) and measured (blue bars) free target concentrations from target: drug complexes when Mab-1, Mab-2 or Mab-3 were used as capture antibodies, according to an exemplary embodiment

FIG. 3D shows free target recovery (%AR) from target:drug complexes with molar ratio at a 1:0.25, when the 1:50 diluted samples were incubated with Mab-1, Mab-2 or Mab-3 for approximately 15 or 45 minutes, respectively, according to an exemplary embodiment.

FIG. 3E shows free target recovery with target:drug complexes (with a 1:50 sample dilution) for a sample that underwent one or seven freeze/thaw cycles with Mab-3 as the capture antibody, according to an exemplary embodiment.

FIG. 4 shows a schematic representation of the free target assay with different capture antibodies, according to an exemplary embodiment.

FIG. 5A shows drug, total and free target concentrations in human serum and plasma samples from individuals participating in a single-dose clinical study with 1 mg/kg iv drug, according to an exemplary embodiment.

FIG. 5B shows drug, total and free target concentrations in human serum and plasma samples from individuals participating in a single-dose clinical study with 30 mg/kg iv drug, according to an exemplary embodiment.

FIG. 5C shows drug, total and free target concentrations in human serum and plasma samples from individuals participating in a single-dose clinical study with 300 mg/kg sc drug, according to an exemplary embodiment.

### DETAILED DESCRIPTION

Reliable bioanalytical methods for the measurement of mAbs and their targets in circulation are critical for the assessment of exposure-response relationships in support of efficacy and safety evaluations and dose selection. See Lee, J.W., et al., AAPS J, 2011. 13(1): p. 99-110; Lee, J.W. and H. Salimi-Moosavi. Bioanalysis, 2012. 4(20): p. 2513-23; Zheng, S., T. McIntosh, and W. Wang. J Clin Pharmacol, 2015. 55 Suppl 3: p. S75-84; Gupta, S., et al., 2017 (Part 3 - LBA: immunogenicity, biomarkers and PK assays). Bioanalysis, 2017. 9(24): p. 1967-1996; Yang, J. and V. Quarmby. Bioanalysis, 2011. 3(11): p. 1163-5.

Free mAb levels provide information about the active drug available to bind targets, while total mAb levels can help characterize the dynamic interaction between mAbs and targets. Target concentrations are also used in the nonclinical development stage to determine the efficacious mAb concentration and to allow the model-based determination of dose. Betts, A.M., et al. J Pharmacol Exp Ther, 2010. 333(1): p. 2-13.

Target data in the clinical stage are used to characterize human PK profiles, define PK/pharmacodynamic (PD) relationships regarding safety and efficacy, and establish PK/PD models in the target population. In particular, total target data provides information on the effect of mAbs on target accumulation and whether there is continuous target engagement in circulation to achieve sustained complete target suppression. Monitoring free targets during dosing is informative for determining the efficacious dose and guiding dose level/schedule selection. Free targets can also be used for PK/PD modeling and to help understanding other PD/end point results.

The present invention provides a total drug assay with a mild acidic assay pH to mitigate target interference. The present invention also presents a similar mild acidic assay condition for total target assay together with a pair of anti-target antibodies that have a lower dissociation rate to the target compared to the drug, and are with a much great t_{½} under the acidic assay condition. The present invention also provides a free target assay that was also developed with a capture antibody that has a much lower affinity to the target compared to the drug and exhibits a much slower association rate. These assays thus can accurately quantify drug and target concentrations in clinical study samples, which supports the PK/PD modeling of biotherapeutic drugs.

Unless described otherwise, all technical and scientific terms used herein have the same meaning as commonly understood by one of ordinary skill in the art to which the present invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

As used herein, the term “protein” includes any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides’ refers to a non-naturally occurring peptide or polypeptide. Synthetic peptides or polypeptides can be synthesized, for example, using an automated polypeptide synthesizer. Various solid phase peptide synthesis methods are known. A protein may contain one or multiple polypeptides to form a single functioning biomolecule. A protein can include any of biotherapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. In another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a review discussing biotherapeutic proteins and their production, see Ghaderi et al., “Production platforms for biotherapeutic glycoproteins. Occurrence, impact, and challenges of non-human sialylation,” (BIOTECHNOL. GENET. ENG. REV. 147-175 (2012)). In some exemplary embodiments, proteins comprise modifications, adducts, and other covalently linked moieties. Those modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) mycepitope, fluorescent labels and other dyes, and the like. Proteins can be classified on the basis of compositions and solubility and can thus include simple proteins, such as, globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein of interest can be an antibody, a bispecific antibody, a multispecific antibody, antibody fragment, monoclonal antibody, or an Fc fusion protein.

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H2} and C_{H}3. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different exemplary embodiments, the FRs of the anti-big-ET-1 antibody (or antigen-binding portion thereof) may be identical to the human germline sequences, or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab’ fragment, a F(ab')2 fragment, a Fc fragment, a scFv fragment, a Fv fragment, a dsFv diabody, a dAb fragment, a Fd' fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. An antibody fragment may be produced by various means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

The term “Fc fusion proteins” as used herein includes part or all of two or more proteins, one of which is an Fc portion of an immunoglobulin molecule, that are not fused in their natural state. Preparation of fusion proteins comprising certain heterologous polypeptides fused to various portions of antibody-derived polypeptides (including the Fc domain) has been described, e.g., by Ashkenazi et al., Proc. Natl. Acad. Sci USA 88: 10535, 1991; Byrn et al.,Nature 344:677, 1990; and Hollenbaugh et al.,“Construction of Immunoglobulin Fusion Proteins,” in Current Protocols in Immunology, Suppl. 4, pages 10.19.1-10.19.11, 1992. “Receptor Fc fusion proteins” comprise one or more of one or more extracellular domain(s) of a receptor coupled to an Fc moiety, which in some embodiments comprises a hinge region followed by a CH2 and CH3 domain of an immunoglobulin. In some embodiments, the Fc-fusion protein contains two or more distinct receptor chains that bind to a single or more than one ligand(s). For example, an Fc-fusion protein is a trap, such as for example an IL-1 trap (e.g., Rilonacept, which contains the IL-1 RAcP ligand binding region fused to the IL-1R1 extracellular region fused to Fc of hIgG1; see U.S. Pat. No. 6,927,004, which is herein incorporated by reference in its entirety), or a VEGF Trap (e.g., Aflibercept, which contains the Ig domain 2 of the VEGF receptor Flt1 fused to the Ig domain 3 of the VEGF receptor Flk1 fused to Fc of hIgG1; e.g., SEQ ID NO: 1; see U.S. Pat. Nos. 7,087,411 and 7,279,159, which are herein incorporated by reference in their entirety).

As used herein, the term “Affinity” refers to the strength of interaction between protein of interest or protein and target. The affinity is measured in K_{D}.

The term “sample” as used herein includes any biological specimen obtained from a patient. Samples include, without limitation, whole blood, plasma, serum, red blood cells, white blood cells (e.g., peripheral blood mononuclear cells), saliva, urine, stool (i.e., feces), sputum, bronchial lavage fluid, tears, nipple aspirate, lymph (e.g., disseminated tumor cells of the lymph node), fine needle aspirate, any other bodily fluid, a tissue sample (e.g., tumor tissue) such as a biopsy of a tumor (e.g., needle biopsy), and cellular extracts thereof. In some embodiments, the sample is whole blood or a fractional component thereof such as plasma, serum, or a cell pellet. In preferred embodiments, the sample is obtained by isolating circulating cells of a solid tumor from whole blood or a cellular fraction thereof using any technique known in the art and preparing a cellular extract of the circulating cells. In other embodiments, the sample is a formalin fixed paraffin embedded (FFPE) tumor tissue sample, e.g., from a solid tumor of the lung, colon, or rectum.

In other embodiments, the sample can comprise of whole blood, serum, plasma, urine, sputum, bronchial lavage fluid, tears, nipple aspirate, lymph, saliva, and/or fine needle aspirate sample. In certain instances, the whole blood sample is separated into a plasma or serum fraction and a cellular fraction (i.e., cell pellet). The cellular fraction typically contains red blood cells, white blood cells, and/or circulating cells of a solid tumor such as circulating tumor cells (CTCs), circulating endothelial cells (CECs), circulating endothelial progenitor cells (CEPCs), cancer stem cells (CSCs), and combinations thereof. The plasma or serum fraction usually contains, inter alia, nucleic acids (e.g., DNA, RNA) and proteins that are released by circulating cells of a solid tumor.

As used herein, the term “capture reagent” is used herein to refer to binding reagents that are immobilized on surface to form a binding surface for use in the assay. The assay modules and methods may also employ or include another binding reagent, “the detection reagent” whose participation in binding reactions on the binding surface can be measured. The detection reagents may be measured by measuring an intrinsic characteristic of the reagent such as color, luminescence, radioactivity, magnetic field, charge, refractive index, mass, chemical activity, etc. Alternatively, the detection reagent may be labeled with a detectable label and measured by measuring a characteristic of the label. Suitable labels include, but are not limited to, labels selected from the group consisting of electrochemiluminescence labels, luminescent labels, fluorescent labels, phosphorescent labels, radioactive labels, enzyme labels, electroactive labels, magnetic labels and light scattering labels.

The capture or detection reagents may directly bind to (or compete with) an analyte (drug or target) of interest or may interact indirectly through one or more bridging ligands. Accordingly, the dry assay reagents may include such bridging ligands. By way of example, streptavidin or avidin may be used as capture or detection reagents by employing biotin-labeled bridging reagents that bind or compete with the analyte of interest. Similarly, anti-hapten antibodies may be used as capture or detection reagents by employing hapten labeled binding reagents that bind or compete with the analyte of interest. In another example, anti-species antibodies or Fc receptors (e.g., Protein A, G or L) are used as capture or detection reagents through their ability to bind to analyte specific antibodies. Such techniques are well established in the art of binding assays and one of ordinary skill in the art will be able to readily identify suitable bridging ligands for a specific application.

Certain embodiments of the assay modules/plates include a capture reagent immobilized on a surface of the module/plate so as to form a binding surface. Immobilization may be carried out using well established immobilization techniques in the art of solid phase binding assays such as the techniques that have been established for carrying out ELISA assays or array-based binding assays. In one example, binding reagents may be non-specifically adsorbed to a surface of a well of a multi-well plate. The surface may be untreated or may have undergone treatment (e.g., treatment with a plasma or a charged polymer) to enhance the adsorbance properties of the surface. In another example, the surface may have active chemical functionality that allows for covalent coupling of binding reagents. After immobilizing the reagent, the surface may, optionally, be contacted with a reagent comprising a blocking agent to block uncoated sites on the surface. For conducting multiplexed measurements, binding surfaces with arrays of different capture reagents may be used. A variety of techniques for forming arrays of capture reagents are now well established in the art of array-based assays.

The binding surfaces are, optionally, coated with a reconstitutable dry protective layer. The protective layer may be used to stabilize a binding surface, to prevent a binding surface from contacting detection reagents during manufacture or storage, or simply as a location to store assay reagents such as bridging reagents, blocking reagents, pH buffers, salts, detergents, electrochemiluminescence coreactants, etc. Stabilizers that may be found in the protective layer include, but are not limited to, sugars (sucrose, trehalose, mannitol, sorbitol, etc.), polysaccharides and sugar polymers (dextran, FICOLL, etc.), polymers (polyethylene glycol, polyvinylpyrrolidone, etc.), zwitterionic osmolytes (glycine, betaine, etc.) and other stabilizing osmolytes (trimethylamine-N-oxide, etc.). Blocking agents are materials that prevent non-specific binding of assay components, especially detection reagents, to binding surfaces and include proteins (such as serum albumins, gamma globulins, immunoglobulins, dry milk or purified casein, gelatin, etc.), polymers (such as polyethylene oxide and polypropylene oxide) and detergents (e.g., classes of non-ionic detergents or surfactants are known by the trade names of BRIJ™, TRITON™, TWEEN®, THESIT®, LUBROL, GENAPOL®, PLURONIC®, TETRONIC®®, and SPAN). In certain embodiments, a protective layer is included that comprises ammonium phosphate as a buffering component, comprises other ammonium salts, and/or comprises less than 1% or 0.1% (w/w) sodium or potassium ions.

The solid support can comprise any suitable substrate for immobilizing proteins. Examples of solid supports include, but are not limited to, glass (e.g., a glass slide), plastic, chips, pins, filters, beads (e.g., magnetic beads, polystyrene beads, etc.), paper, membranes, fiber bundles, gels, metal, ceramics, and the like. Membranes such nylon (Biotrans™, ICN Biomedicals, Inc. (Costa Mesa, Calif.); Zeta-Probe®, Bio-Rad Laboratories (Hercules, Calif.)), nitrocellulose (Protran®, Whatman Inc. (Florham Park, N.J.)), and PVDF (Immobilon™, Millipore Corp. (Billerica, Mass.)) are suitable for use as solid supports in the arrays of the present invention. Preferably, the capture antibodies are restrained on glass slides coated with a nitrocellulose polymer, for example, FAST® Slides, which are commercially available from Whatman Inc. (Florham Park, N.J.).

The term “incubating” is used synonymously with “contacting” and “exposing” and does not imply any specific time or temperature requirements unless otherwise indicated.

Various publications, including patents, patent applications, published patent applications, accession numbers, technical articles and scholarly articles are cited throughout the specification. Each of these cited references is incorporated by reference, in its entirety.

The present invention will be more fully understood by reference to the following Examples. They should not, however, be construed as limiting the scope of the invention.

### EXAMPLES

Materials and Reagents. For the total drug assay, the total target assay, and the free target assay, all solutions, unless otherwise specified, were prepared in assay dilution buffer (0.5% bovine serum albumin [BSA], 0.05% Tween-20, 1× phosphate-buffered saline [PBS]) PBS was from Gibco (Grand Island, NY). Glacial acetic acid was from Thermo Fisher Scientific (Waltham, MA). Human serum and plasma were from BioIVT (Westbury, NY). Streptavidin-coated microplates were from Meso Scale Discovery (MSD; Rockville, MD). Black microwell plates, NeutrAvidin conjugated with horseradish peroxidase (NeutrAvidin-HRP), and SuperSignal ELISA Pico Chemiluminescent Substrate were from Thermo Fisher Scientific (Rockford, IL). Purified native human target was from EMD Millipore (Burlington, MA). Drug (a fully human mAb), mouse anti-drug mAb, biotinylated mouse anti-drug mAb, all human anti-target mAbs (used in the target assays) were produced by Regeneron Pharmaceuticals (Tarrytown, NY).

Total Drug Assay. This assay includes a mild acid treatment of serum samples to dissociate soluble target:drug complexes and improve detection of drug while soluble target is present in the serum. The procedure employs a microtiter plate coated with a mouse anti-drug mAb (2 µg/mL) and utilizes Drug as a standard. The standards, controls, and samples were diluted 1:50 in 30 mM acetic acid in ADB (pH ~5.0), and were added to the plate. Drug captured on the plate was detected using a different, noncompeting, biotinylated mouse anti-drug mAb (200 ng/mL), followed by NeutrAvidin-HRP (200 ng/mL). All incubations were performed at room temperature for approximately 60 minutes. Finally, a luminol-based substrate specific for peroxidase was then added to achieve a signal intensity that is proportional to the concentration of total Drug.

Total Target Assay. This assay includes mild acid pre-treatment of plasma samples to dissociate soluble target:drug complexes present in the plasma samples and improve detection of the target in the presence of drug. The procedure employs a streptavidin-coated MSD plate, with a biotinylated human anti-target mAb (5 µg/mL) as the capture reagent and utilizes purified target as a standard. The standards, controls, and samples (samples with a 1:5 pre-dilution in 5% BSA) were diluted 1:10 in 30 mM acetic acid in 5% BSA (pH about 6.0; 5% BSA was used to reduce assay background) and then further diluted 1:5 in 30 mM acetic acid in 5% BSA containing 6.25 µg/mL of the capture mAb prior addition to the plate. After the 1:5 dilution, the final concentration of the capture mAb in the Sample/Capture Antibody mixture was 5 µg/mL Target bound to the capture reagent and then captured on the plate was detected using a different ruthenium-labeled human anti-target mAb (2 µg/mL). All incubations were performed at room temperature for approximately 60 min. Finally, target concentrations were measured by an electrochemiluminescent signal generated by the ruthenium label when voltage was applied to the plate by the MSD plate reader. The resulting electrochemiluminescent signal (e.g., counts) is proportional to the concentration of total target in the plasma samples.

Free Target Assay. The procedure employs a streptavidin-coated MSD plate, with a biotinylated human anti-target mAb (5 µg/mL) as the capture reagent and utilizes purified target as a standard. The standards, quality controls (QCs) and samples were diluted either 1:2 or 1:50 in 5% BSA and were then added to the plates. Plates were incubated at room temperature for approximately 15 or 45 minutes. Target bound to the capture mAb and then captured on the plate, was detected using a different ruthenium-labeled human anti-target mAb (2 µg/mL) that does not compete with the drug or the different capture antibodies for target binding. Plates were incubated at room temperature for approximately 15 minutes. Finally, target concentrations were measured by an electrochemiluminescent signal generated by the ruthenium label when voltage was applied to the plate by the MSD plate reader. The resulting electrochemiluminescent signal (e.g., counts) is proportional to the concentration of free target in the plasma samples.

Preparation of Target:Drug Complexes. Target:drug complexes at molar ratios of 1:5, 1:2, 1:1,1:0.5,1:0.25 and 1:0.125 were prepared in a naive human plasma sample with known target concentration based on analysis using the total target assay. The target: drug complex samples were prepared by spiking specific amounts of drug into the plasma sample based on the molecular weight of the target and the drug. The resulting target: drug mixtures were incubated at room temperature for approximately 1 to 3 hours before being used in the target assays.

Biacore surface plasmon resonance analysis. Binding kinetics and affinities for anti-target antibodies were assessed using surface plasmon resonance technology on a Biacore T200 (Cytiva, MA, USA) instrument using a Series S CM5 sensor chip in filtered and degassed PBS-T running buffer (0.01 M Na2HPO4/NaH2PO4, 0.15 M NaCl, 0.05% v/v Tween-20, at pH 7.4 or pH 6.0). The capture sensor surfaces were prepared by covalently immobilizing with a mouse anti-human Fc mAb using the standard amine coupling chemistry as previously reported. Different concentrations of target (prepared in PBS-P, pH 7.4 running buffer ranging from 100 to 11.11 nM, threefold dilutions) were injected over the anti-target antibody captured surface for 3 min at a flow rate of 50 pl/min, and their dissociation in two running buffers PBS-T, pH about 7.4 and PBS-T, pH 6.0 was monitored for 6 min. At the end of each cycle, the anti-human Fc surface was regenerated using a 12-s injection of 20 mM phosphoric acid. All of the specific surface plasmon resonance binding sensorgrams were double-reference subtracted as previously reported. Kinetic parameter constants (kd and ka) were determined by fitting the real-time sensorgrams to a 1:1 binding model using Scrubber 2.0 c (BioLogic Software, Campbell, Australia) curve fitting software. The dissociation rate constant (kd) was determined by fitting the change in the binding response during the dissociation phase, and the association rate constant (ka) was determined by globally fitting analyte binding at different concentrations. The equilibrium dissociation constant (KD) was calculated from the ratio of the kd and ka. The dissociative half-life (t1/2) in minutes was calculated as ln2/(kd*60).

### Example 1. Use of Mild Acidic Assay pH to Mitigate Target Interference in the Total Drug Assay

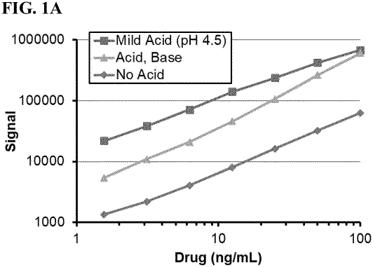

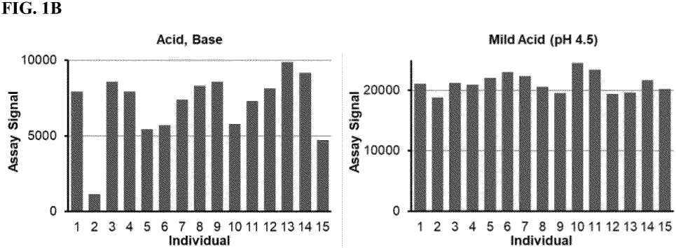

A sandwich ELISA assay format, with two non-competing anti-drug antibodies as the capture and detection reagents, was investigated to quantitate total drug concentrations. Initial experiments with standards prepared in human serum generated unexpectedly poor signals (FIG. 1A), much lower than standards prepared in monkey serum (data not shown). This result suggested that the drug target, present at high concentrations in serum (80-100 µg/mL) bound to the drug, may be interfering with the ability of the anti-idiotype capture or detection mAb to recognize the drug.

To minimize potential target interference, serum samples were acidified (300 mM acetic acid) to dissociate target:drug complexes and then neutralized before analysis. The acid pre-treatment step followed by neutralization significantly improved the assay signal, although not uniformly throughout the range of the standard curve. At the ULOQ, acid pre-treatment increased signal by approximately about 10-fold, whereas at the LLOQ, the signal increased by approximately about 4-fold (FIG. 1A). In addition, the assay generated variable signal responses with individual serum samples spiked at the LLOQ (FIG. 1B), suggesting that endogenous target levels may interfere at the low end of the calibration range.

Published data indicate that under mild acidic assay conditions (pH about 5), some targets can dissociate from the drug while anti-idiotype mAbs retain binding ability (Partridge, M.A., et al., Minimizing target interference in PK immunoassays: new approaches for low-pH-sample treatment. Bioanalysis, 2013. 5(15): p. 1897-910.). To test this approach in total drug assay, standards in human serum were diluted in mild acetic acid (30 mM) and analyzed in the sandwich ELISA without a neutralization step. As with standard acidification/neutralization pre-treatment, assay signal for samples diluted in mild acid increased significantly compared to samples analyzed without acid pre-treatment. However, with the mild acid approach, the improvement in assay signal was uniform throughout the range of the standard curve (FIG. 1A). In addition, the assay generated more consistent signal responses with individual serum samples spiked at the LLOQ level (FIG. 1B), with %AR within ± 20% of the nominal spiked drug concentration for all spiked samples (data not shown).

### Example 2. Capture and Detection Antibody Selection and Use of Mild Acidic Assay pH to Mitigate Drug Interference in the Total Target Assay

The target concentration in naive human plasma samples is approximately 80-100 µg/mL and may be higher in post-dose samples from clinical studies. Therefore, in order to achieve sustained target suppression, significant amounts of drug are usually administered. As such, a significant level of target: drug complexes are usually formed in circulation, which may in turn impact the accurate detection of the total target. Two anti-target antibodies that do not compete with drug for target binding were initially selected as the capture and detection reagents to measure the total target (Table 1).

TABLE 1

Characterization of the Original and the New Capture and Detection Antibodies for the Total Target Assay

| | pH ~7.0 | | | pH ~6.0 | |
|---|---|---|---|---|---|
| | K_{D} (M) | k_{d} (⅟s) | t_{½} (min) | k_{d} (⅟s) | t_{½} (min) |
| Drug | 2.9E-11 | 2.8E-05 | 415 | 1.3E-04 | 92 |
| New Capture | 2.3E-11 | 1.0E-05* | 1155 | 1.0E-05* | 1155 |
| New Detection | 2.8E-11 | 1.0E-05* | 1155 | 1.0E-05* | 1155 |
| Original Capture | 2.6E-10 | 4.5E-05 | 260 | 7.0E-05 | 165 |
| Original Detection | 1.8E-10 | 6.2E-05 | 186 | 1.0E-04 | 114 |

*Under the current experimental conditions, no dissociation of target was observed from the captured monoclonal antibody and k_{d} value was fixed at 1.0E-05.

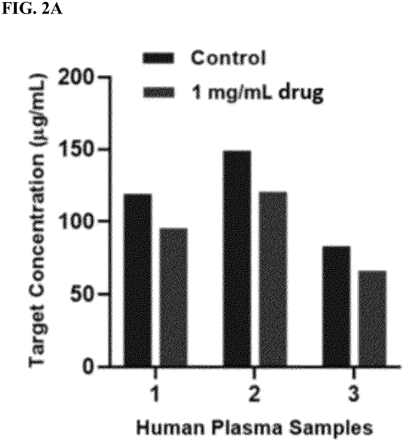

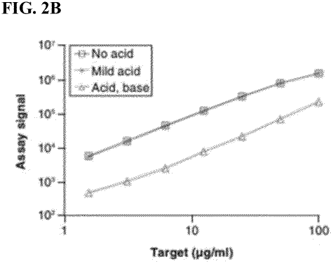

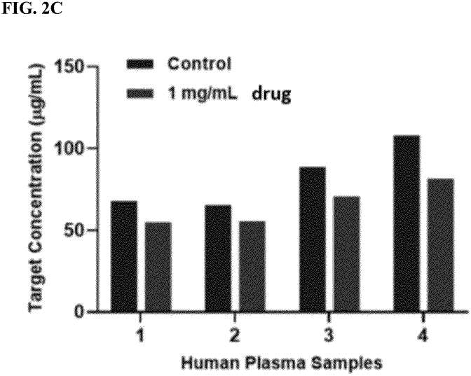

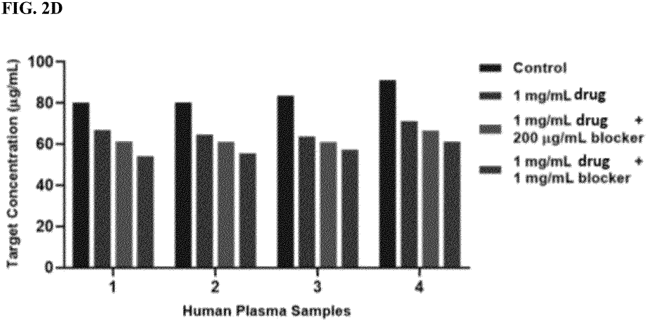

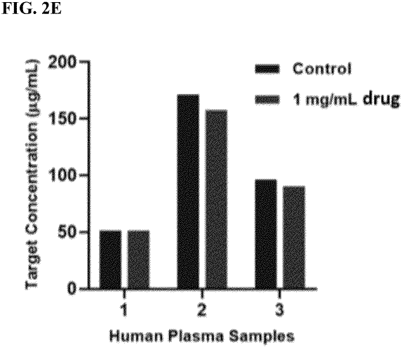

Total target levels in three naive human plasma samples ranged from 80 to 150 µg/mL (FIG. 2). However, the target concentrations decreased approximately 20% in the presence of 1 mg/mL of drug (FIG. 2A), suggesting that, even though two anti-target antibodies that do not compete with drug for target binding, steric hindrance may impact the binding of either the capture and/or the detection antibody to the target when drug is present at a high concentration. Acid dissociation was then used to dissociate the target:drug complexes. However, the assay signal was greatly reduced after acid treatment (300 mM acetic acid) and neutralization (FIG. 2B) compared with the signal obtained under neutral conditions, suggesting that the target protein may not be stable under this stringent acidic condition. Similar to the total drug assay, a mild acidic assay condition (pH about 6.0) was then used to dissociate the target:drug complexes. The standard curve signal with the mild acidic pH was comparable to the signal with no acid treatment (FIG. 2B). However, in the presence of 1 mg/mL of Drug, a 15% to 25% decrease in target concentrations was still observed (FIG. 2C).

Anti-drug antibodies have been successfully used to mitigate drug interference in the determination of the total target concentrations (unpublished data). An anti-drug antibody that can effectively block target and drug binding was tested in the total target assay with the mild acidic assay pH (pH about 6.0). Neither 200 µg/mL nor 1 mg/mL of the anti-drug antibody was able to effectively inhibit drug interference under mild acidic assay conditions (FIG. 2D), probably because the mild acidic assay condition was not able to fully dissociate the target:drug complexes and/or the antibody may not be effective in binding to the drug under this acidic assay condition.

The total target assay was re-developed (second generation assay) using two new anti-target antibodies as the capture and detection reagents. The two anti-target antibodies selected have a lower dissociation rate to the target and a much greater t_{½} at pH ~6.0 when compared to the drug (Table 1). In fact, under the mild acidic pH, the dissociation rate of drug increases approximately 5-fold, with a much shorter t_{½} when compared to that from the neutral pH. These results indicate that, under acidic conditions, when the binding of the drug to the target is greatly reduced, both the new capture and detection antibody can still effectively bind to the target. Finally, similar target concentrations were obtained in ten human plasma samples with the second-generation assay format (FIG. 2E), in the absence and presence of 1 mg/mL of drug, indicating the drug interference was successfully mitigated with the new capture and detection antibody pair and with mild acidic assay pH (pH ~6.0).

### Example 3. Capture Antibody Selection and Assay Format Optimization in the Development Of the Free Target Assay

Since free target data are useful in determining the efficacious dose and guiding the dose selection, a free target assay was also developed to support this program. In order to accurately measure the free target concentration, particular considerations must be made such as capture antibody selection, sample dilution, sample incubation time and the stability of the target:drug complexes. See (Hansen, R.J., et al. MAbs, 2013. 5(2): p. 288-96; Liu, Y., et al., Bioanalysis, 2021. 13(7): p. 575-585; Colbert, A., et al. MAbs, 2014. 6(4): p. 1103-13; Peng, K., et al. AAPS J, 2018. 21(1): p. 9.). Three anti-target antibodies that compete with drug for target binding, Mab-1, Mab-2 and Mab-3, were tested as the capture antibody in the free target assay. Mab-2 has a similar K_{D} value to the drug, while Mab-1 and Mab-3 have approximately 5-7 fold lower affinity to the target compared to the drug (Table 2).

TABLE 2

Characterization of Antibodies Mab-1, Mab-2 and Mab-3 for the Free Target assay

| | | | |
|---|---|---|---|
| | kₐ (⅟Ms) | k_{d} (⅟s) | K_{D} (M) |
| Drug | 9.8E+05 | 2.8E-05 | 2.9E-11 |
| Mab-1 | 9.7E+05 | 1.4E-04 | 1.4E-10 |
| Mab-2 | 1.1E+06 | 5.5E-05 | 4.9E-11 |
| Mab-3 | 5.3E+04 | 1.0E-05* | 1.9E-10 |

Target:drug complexes at a 1:1, 1:2, and 1:5 molar ratio were diluted 1:2 prior to analysis to prevent any potential dissociation of the target:drug complexes. In complexes where drug is in excess (e.g., 1:5 and 1:2 molar ratios), little to no detectable levels of free target are expected, therefore, assay signal from these complexes should be at or below the assay low limit of quantitation signal (LLOQ, 1.56 µg/mL).

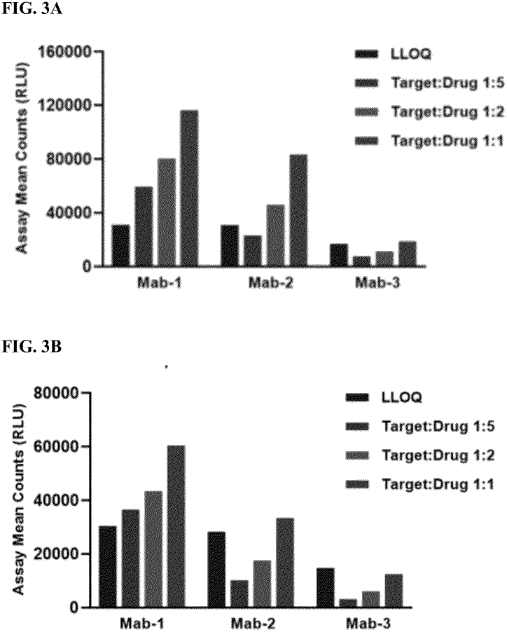

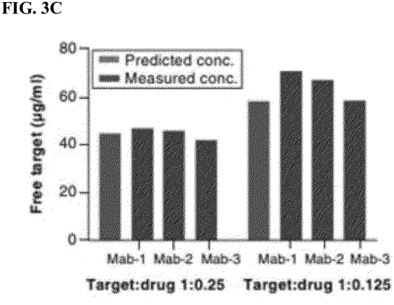

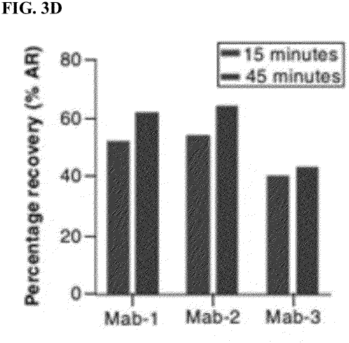

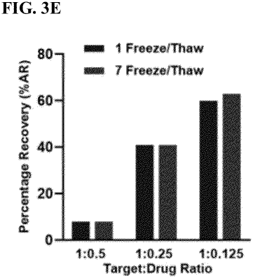

When Mab-1 or Mab-2 was used as the capture antibody, the assay signal from these complexes were either greater than the assay LLOQ or close to the LLOQ (FIG. 3A), indicating higher concentrations of free target were being measured. However, when Mab-3 was used as the capture reagent, the assay signal from these complexes was below the assay LLOQ signal (FIG. 3A). Although a small amount of free target may be detected when target and drug are present in equimolar concentrations (e.g., 1:1 complex), a much higher signal was observed with Mab-1 and Mab-2 when compared to Mab-3. These results suggest that, as target dissociates from drug in solution, Mab-1 and Mab-2 (with higher association rates for the target than Mab-3) may more effectively compete with the drug and capture the free target on the plate surface, removing it from the solution, preventing its reassociation with the drug and further disrupting the equilibrium.

To further evaluate these capture antibodies and to ensure samples can be further diluted so their signals are within the range of the standard curve, a greater sample dilution (1:50) was performed to quantitate the free target in the presence of different concentrations of the drug. Similar to the previous findings, when Mab-1 was used as the capture antibody, signal from these target:drug complexes was above the assay LLOQ signal (FIG. 3B), indicating there is still dissociation of the complex with at this higher sample dilution. The assay signal from the 1:1 complex was above the assay LLOQ when Mab-2 was used as the capture reagent. However, assay signal from the 1:5, 1:2 and 1:1 complexes was below assay the LLOQ signal when Mab-3 was used as the capture antibody (FIG. 3B), suggesting this assay format may be able to more accurately measure the free target concentrations present in the samples.

Free target concentrations were also measured with the target:drug complexes at 1:0.5, 1:0.25, and 1:0.125 molar ratios, when measurable level of free target are expected. The amount of free target detected with Mab-3 as the capture antibody was very similar to the free target concentrations predicted based on a target-mediated drug disposition PK model developed using total drug and total target concentrations in clinical settings (FIG. 3C). However, the concentrations measured with Mab-1 or Mab-2 as capture antibodies were greater than the model prediction (FIG. 3C), further suggesting that these antibodies have a greater impact on the equilibrium and favor target:drug complex dissociation..

Finally, the free target concentration in these complexes was also measured when sample incubation times were varied for approximately 15 or 45 minutes. With Mab-3 as the capture antibody, the free target concentrations were comparable regardless of the sample incubation time (FIG. 3D), indicating there was minimal impact on the complexes in solution even with a longer sample incubation time. However, when Mab-1 or Mab-2 was used as the capture antibody, the free target concentrations increased with longer sample incubation times (FIG. 3D), suggesting further dissociation of the complexes. Finally, similar free target concentrations were obtained when these samples had either one or seven freeze/thaw cycles with Mab-3 as the capture antibody (FIG. 3E), indicating that target:drug complexes and free target were stable in these samples, even when the sample was subject to stress.

Capture antibody selection is critical in the accurate measurement of free target concentrations. See Liu, Y., et al., Development of a Meso Scale Discovery ligand-binding assay for measurement of free (drug-unbound) target in nonhuman primate serum. Bioanalysis, 2021. 13(7): p. 575-585). In this study, Mab-3 has a much lower affinity (K_{D} value) to the target compared with the drug and exhibits a much slower association rate, so it is less likely to disrupt the equilibrium of tragte:drug complexes in solution. Therefore, even though in equilibrium, there is dissociation of target:drug in the 1:5, 1:2 and 1:1 complexes, the majority of the target may quickly re-associate with the drug and is therefore not detected in the free target assay. Only the free target is captured by Mab-3 and detected in the assay (FIG. 4). However, even though Mab-1 also has a lower affinity to the target compared with the drug, it has a similar association rate (kₐ) as the drug. It is possible that some of the dissociated target from target: drug complexes may bind to Mab-1 on the plate instead of re-associating with the drug, thus being detected in the free target assay (FIG. 4) and resulting in overestimation of the free target concentration. The top panel of FIG. 4 shows a schematic representation of the free target assay for when Mab-1 or Mab-2 are used as capture antibodies, because they have a similar ka value as the Drug (even though Mab-1 has a lower affinity) some of the newly dissociated target from target:drug complexes may bind to Mab-1 or Mab-2 on the plate surface, instead of reassociating with the Drug in solution, resulting in overestimation of the free target concentration. The bottom panel shows a schematic representation of when Mab-3, with a lower affinity and a slower association rate with the target compared with the Drug, is used as the capture reagent, newly dissociated target may quickly reassociate with the Drug and is not detected in the free target assay. Only the free target is captured by Mab-3 and detected in the assay.

### Example 4. Measurement of Total Drug, Total and Free Target Concentrations in Clinical Study Samples

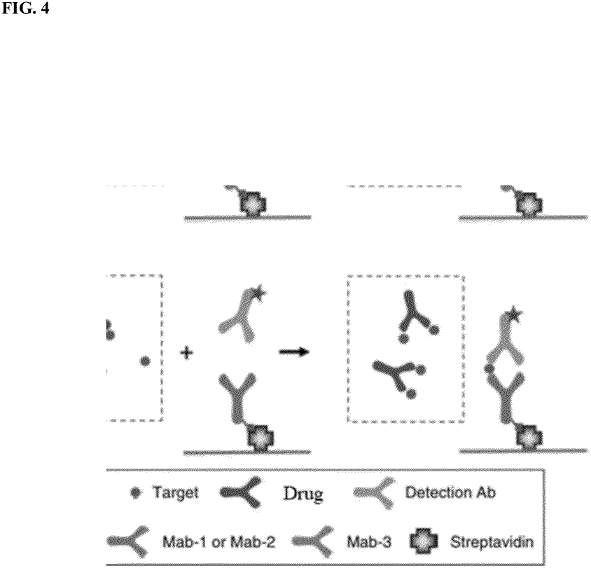

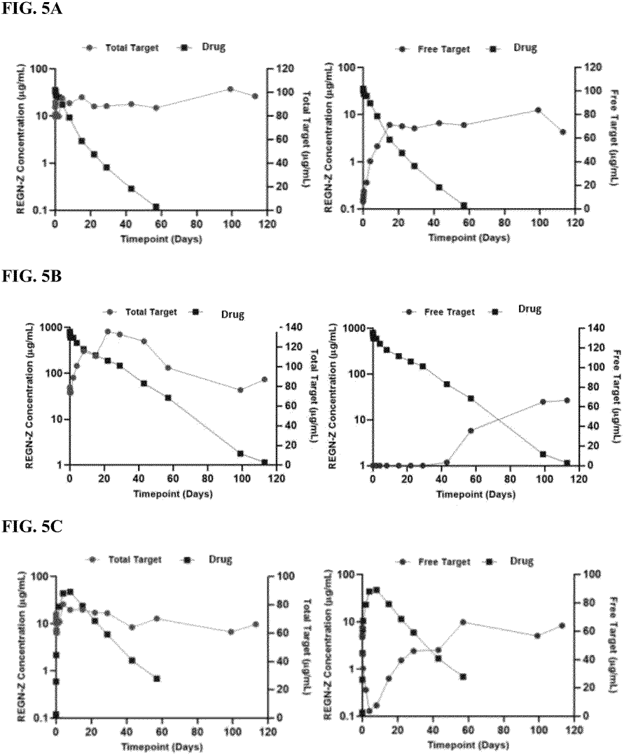

Samples from individuals who received 1 mg/kg Drug intravenously (iv.), 30 mg/kg Drug (iv.), or 300 mg Drug subcutaneously (sc.) in a phase I, single-dose clinical study were tested for total drug concentrations in serum, as well as for total and free target concentrations in plasma (FIG. 5).

For the individual dosed with 1 mg/kg Drug, iv., (FIG. 5A), circulating drug concentrations decreased over time and diminished around Day 57, with total target levels of 80 - 100 µg/mL for all time points tested and no noticeable target accumulation (FIG. 5A). However, free target levels decreased significantly after drug administration and remained at low levels for about one-week post-dose before gradually returning to baseline levels.

For the individual dosed with 30 mg/kg Drug, iv., the drug concentration reached significantly higher levels within the first four weeks, started to decrease around Day 29, and was very low by Day 99 (FIG. 5B). Total target concentration appeared to increase from around Day 2 to Day 4, reached the highest levels at Day 22 to Day 29, and subsequently returned to baseline level (FIG. 5B). Free target was undetectable once the drug was administered until Day 57, when the drug concentration had significantly reduced (FIG. 5B). There was a strong correlation between decreased free target levels and increased Drug concentrations in both the 1 mg/kg and 30 mg/kg doses. Similar correlations were also observed with an individual dosed with 300 mg the drug sc. (FIG. 5C). These data demonstrate that the total and free target assays can accurately quantitate target concentrations in clinical study samples, with results that correlate well with the total drug concentrations.

In this study, a total drug assay, a total and a free target assay were developed to support the development program for the therapeutic mAb, Drug. Because of the high endogenous target concentrations, the drug is administrated at very high concentrations. To mitigate target interference in the total drug assay, a mild acidic assay condition was used. The initial total target assay had drug interference issues which could not be mitigated by mild acidic conditions or the use of an anti-drug antibody. Therefore, the total target assay was re-developed, and a new pair of anti-target antibodies with a (1) much higher affinity to the target compared to the drug and (2) much greater half-life (t_{½}) values under mild acidic conditions were used as the capture and detection reagents to mitigate drug interference. The new assay format can accurately quantify total target concentrations in human plasma samples in the presence of drug. In addition, a free target assay was also developed with a capture antibody that has a much slower association rate to the target compared to the drug. The measurement of free target concentrations can be even more challenging. As shown above, multiple anti-target antibodies, which compete with the drug for target binding, were evaluated in the development of the free target assay. This assay format was not impacted by sample dilution, sample incubation time with the capture antibody, or sample freeze/thaw cycles. Finally, these assays showed accuracy when used to determine the concentration of total drug, total target, and free target in a subset of phase I clinical study samples to demonstrate their accuracy.

What is claimed is:

1. A method for determining concentration of free target in a sample, comprising:
a. adding said sample having a bound target and said free target to a solid support coated with a capture agent, wherein said capture agent has less affinity and a greater t_{½} for the target compared to the drug;
b. adding a detection agent with a detectable label; and
c. measuring a signal from the detectable label of the detection agent to determine said concentration of free target in said sample, whereby the signal is proportional to the concentration of said free target in said sample.

2. The method of claim 1, wherein said solid support is a streptavidin coated.

3. The method of claim 1, wherein the captured agent is biotinylated.

4. The method of claim 1, wherein said sample of (a) is incubated for about 15 or about 45 minutes.

5. The method of claim 1, wherein said detectable label is ruthenium.

6. The method of claim 1, wherein said detectable label is an electrochemiluminescent substrate.

7. The method of claim 1, wherein said signal of (c) is obtained by applying voltage.

8. The method of claim 1, further comprising determining an amount of free target from said signal by comparing the signal to a standard calibration curve, wherein the standard calibration curve is produced by using at least three standard solutions having three different concentrations of said free target instead of said sample.

9. A method for determining concentration of total target in a sample, comprising:
a. contacting said sample to an acid solution;
b. adding said sample to a solid support coated with a capture agent;
c. adding a detection agent with a detectable label; and
d. measuring a signal from the detection agent to determine the concentration of said total target in said sample,
wherein said total target includes target complexed with a drug and free target, and
whereby the signal is proportional to the concentration of said total target in said sample.

10. The method of claim 9, wherein the captured agent is biotinylated.

11. The method of claim 9, wherein said detectable label is ruthenium.

12. The method of claim 9, wherein said detectable label is an electrochemiluminescent substrate.

13. The method of claim 9, wherein said signal of (c) is obtained by applying voltage.

14. The method of claim 9, wherein step (b) is incubated for about 60 minutes.

15. The method of claim 9, wherein step (c) is incubated for about 60 minutes.

16. The method of claim 9, wherein said acid solution has a pH of about 5.0 to about 7.0.

17. The method of claim 9, wherein said acid solution has a pH of about 6.0.

18. The method of claim 9, wherein said acid solution comprises 300 mM acetic acid.

19. The method of claim 9, wherein said capture agent has a lower dissociation rate and greater t_{½} towards said target than said drug.

20. The method of claim 9, wherein said detection agent has a lower dissociation rate and greater t_{½} towards said target than said drug.

21. The method of claim 9 further comprising determining an amount of total target from said signal by comparing it to a standard calibration curve, wherein the standard calibration curve is produced by carrying out the method of claim 9 by using at least three standard solutions having three different concentrations of free target instead of said sample.

22. A method for determining concentration of total drug in a sample, comprising:
a. contacting said sample to an acid solution;
b. adding said sample to a solid support coated with a capture agent;
c. adding a detection agent with a detectable label, wherein said capture agent is different than the capture agent; and
d. measuring a signal from the detection agent to determine the concentration of said total target in said sample,
wherein said total drug includes drug complexed to a target and free drug, and
whereby the signal is proportional to the concentration of said free target in said sample.

23. The method of claim 1, further comprising determining an amount of free drug from said signal by comparing the signal to a standard calibration curve, wherein the standard calibration curve is produced by using at least three standard solutions having three different concentrations of said free drug instead of said sample.

24. The method of claim 22, wherein the captured agent is a monoclonal antibody that binds to said drug.

25. The method of claim 22, wherein said detection agent is different from said capturing agent.

26. The method of claim 22, wherein the detection agent is biotinylated.

27. The method of claim 22 further comprising adding a substrate specific to binding with the detection agent, wherein said substrate bound to the detection agent provides said signal of (d).

28. The method of claim 22, wherein said acid solution has a pH of about 5.0 to about 7.0.

29. The method of claim 22, wherein said acid solution has a pH of about 6.0.

30. The method of claim 22, wherein said acid solution comprises 30 mM acetic acid.
